(19) United States
(12) Patent Application Publication
(10) Pub. No.: US 2024/0410011 A1
(43) Pub. Date: Dec. 12, 2024

(54) IMMUNOPHENOTYPING METHOD FOR SMALL-CELL LUNG CANCER ESTABLISHED BASED ON MULTIDIMENSIONAL ANALYSES

(71) Applicant: Cancer Hospital Chinese Academy of Medical Sciences, Beijing (CN)

(72) Inventors: Lin YANG, Beijing (CN); Zicheng ZHANG, Wenzhou (CN); Meng ZHOU, Wenzhou (CN)

(21) Appl. No.: 18/461,736

(22) Filed: Sep. 6, 2023

(30) Foreign Application Priority Data
Jun. 6, 2023 (CN) 202310657822.6

(51) Int. Cl. C12Q 1/6886; C12Q 1/6806; G16B 40/30

(52) U.S. Cl. CPC C12Q 1/6886; C12Q 1/6806; G16B 20/00; G16B 40/20; G16B 40/30; C12Q 2600/112

(57) ABSTRACT

An immunophenotyping method for small-cell lung cancer established based on multidimensional analyses is provided in the present application, including 7 steps of sample collection, RNA extraction, RNA sequencing, unsupervised hierarchical clustering analysis, construction of CCI analysis models, result verification, and typing determination. Human tumor samples are directly taken from clinical archives, and the whole transcriptome and protein digital spatial conformation are adopted to analyze the microenvironmental features of small-cell lung cancer at the RNA and protein levels, respectively, and the concept of immunophenotyping is proposed, classifying small-cell lung cancer into immune-enriched (IE) and immune-deprived (ID) subtypes; the CCI exponent threshold of 0.4 is set to distinguish the small-cell lung cancer into two subtypes of IE and ID.

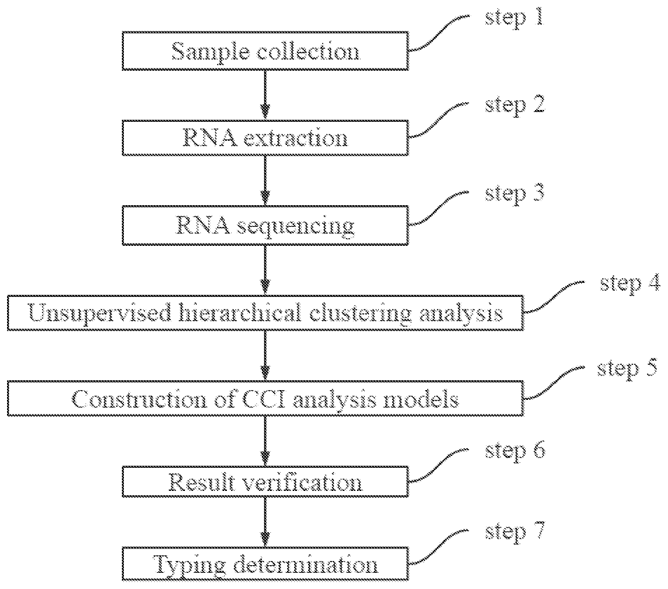

### CROSS-REFERENCE TO RELATED APPLICATIONS

This application claims priority to Chinese Patent Application No. 202310657822.6, filed on Jun. 6, 2023, the contents of which are hereby incorporated by reference.

### TECHNICAL FIELD

The present application relates to the technical field of immunophenotyping of small-cell lung cancer, and in particular to a new immunophenotyping method for small-cell lung cancer established based on multidimensional analyses.

### BACKGROUND

Small-cell lung cancer (SCLC) is a highly malignant lung cancer, for which research has progressed very slowly over the decades, mainly due to the lack of molecular stratification strategies, especially from archived paraffin-embedded samples from routine clinical pathology.

Since 1985, there have been two major findings in the study of SCLC heterogeneity/molecular stratification: (1) Adi F Gazda's subtyping of neuroendocrine differentiation (NE typing), derived mainly from cell line and animal model experiments, based on transcriptome sequencing, which proposed to classify SCLC into NE-High vs. NE-Low (or NE vs. Non-Ne in some literatures) based on high or low expression of a set of 50 genes. The advantage of NE typing is that it reveals for the first time the heterogeneous features of SCLC at the molecular level, which is a major advance after pathomorphological classification, and it is now clear from the review of some proof-of-concept clinical trials (e.g., NCT02484404) that patients with NE typing accompanied by aberrant expression of NOTCH or C-MYC may partially benefit from poly(ADP-ribose) polymerase (PARP) inhibitors or chemotherapy combined with immunotherapy;

NE typing has disadvantages because: a. the samples are mainly from cell lines and animal experiments, which differ from clinical human tumor samples; b. the 50-gene set is derived from transcriptome sequencing, which is difficult to use clinically without a quantitative cut-off; c. 70-80% of clinical SCLC patients are in advanced stages, and advanced stage biopsies are small and few in number, which is unsuitable for transcriptome sequencing analysis; and

other studies based on the concept of NE typing have also proposed studying combinations of several NE markers at the level of protein immunohistochemistry (the traditional three markers Chromogranin A (ChrA), Synaptophysin (Syno), CD56 and the newer one INSM1), but none of them has produced more consistent and accepted results;

(2) molecular typing based on genealogical transcription factors (TF typing), Rudin et al. in 2019 proposed the concept of TF molecular typing based on experimental data from some of their own cell lines plus clustering analysis of transcriptome sequencing data from fresh human tumor samples from George et al. in 2015, i.e. the four subtypes of SCLC-A, SCLC-N, SCLC-Y and SCLC-P; however, subsequent single-cell sequencing studies did not identify the SCLC-Y subtype and the Inflamed subtype was proposed. In addition, TF typing has controversial results for prognostic stratification, and the clinical use of drugs is limited to experimental studies of cell lines, which is far from clinical application; therefore, the present application proposes a new immunophenotyping method for small cell lung cancer based on multidimensional analyses to solve the problems existing in the prior art.

### SUMMARY

In view of the above problems, an objective of the present application is to propose a new immunophenotyping method for small-cell lung cancer (SCLC) established based on multidimensional analyses, and the immunophenotyping method for SCLC established based on multidimensional analyses can be applied directly in the clinical practice, to solve the problems existing in the prior art.

To achieve the objective, the present application includes following technical schemes:
an immunophenotyping method for SCLC established based on multidimensional analyses, including following steps:
step 1, sample collection:
several sets of high-quality formalin-fixed and paraffin-embedded tumor tissues of limited-stage SCLC not treated with chemotherapy and radiotherapy prior to surgery are collected from archived electronic medical record system of a hospital as study samples, and the samples collected are evenly divided into an analytical sample set as well as a validation sample set;
step 2, RNA extraction:
based on the analytical sample set, on each of its sample blocks, several sets of sections are cut and total RNA is isolated from the sections using a paraffin-embedded tissue total RNA extraction kit, and then quantified using a NanoDrop spectrophotometry method, along with a 2100 Bio analyzer for quality control, followed by extraction of RNA from each sample using a Nano String nCounter system;
step 3, RNA sequencing:
tissue microarrays are constructed from SCLC resected from the analytical sample set, by selecting two representative regions of tumor tissue per case, followed by sequencing on an illumination sequencing platform;
step 4, unsupervised hierarchical clustering analysis:
several sets of multi centered SCLC patient cohorts are collected from high-throughput gene expression and corresponding publications to obtain clinicopathological information on SCLC patients in the SCLC patient cohorts, followed by functional and gene enrichment analyses, then unsupervised coherent clustering analysis is applied to molecular data from SCLC tumor samples, and potential molecular subtypes with clustering numbers of 2-5 are identified;
step 5, construction of a cell based computational (CCI) analysis model:
CCI analysis model is constructed using two methods based on genetic characterization of immune cells, including xCell method and single-sample gene set enrichment analysis (ssGSEA) method, and then CCI analysis model is constructed using extreme gradient augmented machine learning algorithms with an upper threshold in a training cohort defined to be 0.4 in a case of CCI specified on a 0-1 exponential.
step 6, result verification:
the validation sample set in step 1 is used as a baseline to measure protein expressions of CCL5 and CXCL9 using quantitative computerized immunohistochemistry (IHC) analysis for experimental validation at a protein level, and the CCI analysis model in step 5 is analytically validated in terms of stability and reliability; and
step 7, typing determination:
based on results of step 6, the CCI analysis model is confirmed in terms of stability and reliability, then the CCI analysis model is applied to a new SCLC immunophenotyping.

A further improvement is that: in the step 1, an inclusion criterion for sample analysis is: after radical cancer surgery coupled with systemic lymph node dissection, histologically confirmed as pure small-cell lung cancer without a component of composite non-small-cell lung cancer, no history of other malignant tumors, and no coexisting tumors in other organs.

A further improvement is that: in the step 2, additional tissue sections of each sample are subjected to hematoxylin and eosin (H&E) staining for pathological verification of tumor areas and borders for macroscopic dissection prior to RNA extraction.

A further improvement is that: in the step 2, an RNA integrity is defined as a percentage of 300 nanograms (ng).

A further improvement is that: in the step 3, data obtained are subjected to quality control (QC) checking and normalization with a QC normalization method used for WTA data merging.

A further improvement is that: in the step 5, the CCI analysis model has a core function of binary logic with a maximum augmentation iteration of 3,000.

A further improvement is that: in the step 6, the CCI analysis model classifies SCLC cases into high CCI group and low CCI group and uses 0.4 as a threshold to represent IE subtype and ID subtype.

A further improvement is that: in the step 6, the CCI analysis model is further characterized for prognostic value in traditional SCLC subtypes by performing stratified analyses in a meta-cohort.

The present application has the following beneficial effects:
this immunophenotyping method for SCLC established based on multidimensional analyses is more relevant to clinical practice than cell line and animal experiment-related tumor microenvironment studies by directly taking human tumor samples from clinical archives, and adopts the whole transcriptome and protein digital spatial conformation to analyze the microenvironmental features of SCLC at the RNA and protein levels, respectively, and proposes the concept of immunophenotyping, which classifies SCLC into immune-enriched (IE) and immune-deprived (ID) subtypes, applicable to both predicting prognosis of limited-stage surgical specimens and assessing the efficacy of immunotherapy for extensive-stage patients; additionally, the CCI exponent threshold of 0.4 can be set to differentiate the SCLC into two subtypes of IE and ID; as such, the present application is more applicable to routine clinicopathological samples, especially in advanced small samples, where the discovery of immunophenotyping can be accomplished by two immunohistochemical markers, CXCL9/CCL5, which is ideally suited for molecular typing of small clinical samples and has a huge advantage over the traditional NE and TF typing.

### BRIEF DESCRIPTION OF THE DRAWING

The FIGURE is a schematic process illustrating steps of the present application.

### DETAILED DESCRIPTION OF THE EMBODIMENTS

To deepen the understanding of the present application, the present application is further described in detail in the following in combination with embodiments, and the embodiments are only used to explain the present application and do not constitute a limitation of the scope of protection of the present application.

### Embodiment 1

As shown in the FIGURE, the present embodiment provides an immunophenotyping method for SCLC established based on multidimensional analyses, including following steps:
step 1, sample collecting:
several sets of high-quality formalin-fixed and paraffin-embedded tumor tissues of limited-stage SCLC not treated with chemotherapy and radiotherapy prior to surgery are collected from archived electronic medical record system of a hospital as study samples, and the samples collected are evenly divided into an analytical sample set as well as a validation sample set; an inclusion criterion for sample analysis is: after radical cancer surgery coupled with systemic lymph node dissection, histologically confirmed as pure small-cell lung cancer without a component of composite non-small-cell lung cancer, no history of other malignant tumors, and no coexisting tumors in other organs;
step 2, RNA extracting:
based on the analytical sample set, on each of its sample blocks, several sets of sections are cut and total RNA is isolated from the sections using a paraffin-embedded tissue total RNA extraction kit, and then quantified using a NanoDrop spectrophotometry method, along with a 2100 Bio analyzer for quality control (QC), followed by extraction of RNA from each sample using a Nano String nCounter system; additional tissue sections of each sample are subjected to hematoxylin and eosin (H&E) staining for pathological verification of tumor areas and borders for macroscopic dissection prior to RNA extraction, and an RNA integrity is defined as a percentage of 300 ng;
step 3, RNA sequencing:
tissue microarrays are constructed from SCLC resected from the analytical sample set, by selecting two representative regions of tumor tissue per case, followed by sequencing on an illumination sequencing platform; data obtained are subjected to QC checking and normalization with a QC normalization method;
step 4, unsupervised hierarchical clustering analysis:
several sets of multicentered SCLC patient cohorts are collected from high-throughput gene expression and corresponding publications to obtain clinicopathological information on SCLC patients in the SCLC patient cohorts, followed by functional and gene enrichment analyses, then unsupervised coherent clustering analysis is applied to molecular data from SCLC tumor samples, and potential molecular subtypes with clustering numbers of 2-5 are identified.
step 5, construction of a CCI analysis model:
CCI analysis model is constructed using two methods based on genetic characterization of immune cells, including xCell method and single-sample gene set enrichment analysis (ssGSEA) method, and then CCI analysis model is constructed using extreme gradient augmented machine learning algorithms with an upper threshold in a training cohort defined to be 0.4 in a case of CCI specified on a 0-1 exponential; the core function of the CCI analysis model is the binary logic, with a maximum augmentation iteration of 3,000;
step 6, result verification:
the validation sample set in the step 1 is used as a baseline to measure protein expressions of CCL5 and CXCL9 using quantitative computerized immunohistochemistry (IHC) analysis for experimental validation at a protein level, and the CCI analysis model in step 5 is analytically validated in terms of stability and reliability; the CCI analysis model classifies SCLC cases into high CCI group and low CCI group and uses 0.4 as a threshold to represent IE subtype and ID subtype; and
step 7, typing determination:
based on results of the step 6, the CCI analysis model is confirmed in terms of stability and reliability, then the CCI analysis model is applied to a new SCLC immunophenotyping.

### Embodiment 2

The present embodiment provides an immunophenotyping method for SCLC established based on multidimensional analyses, including following steps:
step 1, sample collection:
59 sets of high-quality formalin-fixed and paraffin-embedded (FFPE) tumour tissues of limited-stage SCLC that have not received chemotherapy or radiotherapy prior to surgery are selected as study samples from the archival electronic medical record system of the Department of Pathology of the Cancer Hospital Chinese Academy of Medical Sciences (CAMS), and the selected samples are evenly divided into an analytical sample set and a validation sample set, of which, the analytical sample set is 29 sets and the validation sample set is 30 sets; specific inclusion criterion for sample analysis is: after radical cancer surgery coupled with systemic lymph node dissection, histologically confirmed as pure small-cell lung cancer without a component of composite non-small-cell lung cancer, no history of other malignant tumors, and no coexisting tumors in other organs;
step 2, RNA extraction:
the analytical sample set is taken as a baseline, on each of its sample blocks, three sets of sections (8 micrometers (μm) thickness) are cut and total RNA is isolated from the sections using the paraffin-embedded tissue total RNA extraction kit (Qiagen 73504), which is then quantified using the spectrophotometer, along with a bio-analyzer for QC, where the RNA integrity is defined as a percentage of 300 ng, after which RNA from each sample is extracted using custom-designed panel analyses on the Nano String Counter system, which combines the mRNA expression of 277 genes involved in immune checkpoint inhibitors, biomarkers of innate immunity and immune cell types and neuroendocrine traits, after specific probe binding, the gene-specific fluorescent barcodes are hybridized, scanned and quantified on the nCounter FLEX digital analyzer;
QC and raw data processing are conducted using nSolver (v4.0.70), where binding densities between 0.1 and 2.25 are considered good imaging QC and serial dilution spikes with R2 above 0.95 in positive controls are considered good internal QC for quantification.
prior to RNA extraction, additional tissue sections (4 μm thickness) of each sample are subjected to H&E staining (HE staining method) for pathological verification of the tumor areas and borders for macroscopic dissection;
step 3, RNA sequencing:
tissue microarrays (TMAs) are constructed from the analytical sample set, i.e. 29 sets of resected SCLCs, by selecting two representative tumor tissue regions per case, and specifically, the 4 μm TMA sections are deparaffinized and recovered for antigen in Tris-EDTA buffer for 20 minutes (min), RNA target exposure is carried out by incubating the samples with proteinase K, after digestion, the samples are fixed in 10% neutral-buffered formalin (NBF) for 5 min at room temperature, after which probes are applied to tissue sections, with overnight incubation at 37 degrees Celsius (C) in a hybridization chamber, followed by tissue staining with fluorescent antibodies (Pan-CK for epithelial cells, CD45 for immune cells, Nano String) and SYTO 13 (Nuclear Staining, Thermo Fisher) for morphological visualization, and scanned on a digital spatial scanner (Nano String, Seattle, WA, USA), where 1-9 Region of Interests (ROIs) are typically selected, including tumor and intra-tumor stromal regions for each sample, for a total of 83 ROIs, and then UV lysis probes from individual ROIs are collected, assembled, and purified according to the manufacturer's instructions for construction of PCR libraries, followed by QC testing of the PCR products;
sequencing is then performed on the illumination sequencing platform, and the resulting data are subjected to QC and normalized using the QC normalization method used for WTA data merging, and the limit of quantification (LOQ) for each ROI is calculated according to the following formula:

$$LOQ = geomean ( NegProbei ) \times geoSD ( NegProbei ) 2$$

a total of 18,815 genes are included in the downstream analysis and for combined ROI RNA expression comparisons, averages are calculated from normalized individual ROI counts, and in this embodiment, WTA data are overridden to the count level and log 2 (x+1) normalized; and
sequence sections of the same TMAs from 29 resected SCLCs are also analyzed using DSP spatial proteome analysis (Nano String);
step 4, unsupervised hierarchical clustering analysis:
four multicentered cohorts of SCLC patients are collected from Gene Expression Omnibus and corresponding literature, including 50 patients from the GSE60052 cohort, 49 patients from the George study (George cohort), 18 patients from the GSE149507 cohort and 17 anti-PD-1/PD-L1 antibody-treated patients from the Roper study (Roper cohort), and 29 knowledge-based functional gene expression signatures (FGES) from Bagaev's study, covering known immune, stromal and other major cellular functional components of the tumor; clinicopathological information of SCLC patients in the SCLC patient cohort is obtained, 50 signature gene sets are obtained from the Molecular Signature Database (MSigDB, v7.2), and Gene Set Enrichment Analysis (GSEA) is performed by the R package “Cluster Profiler” (v4.2.2);
the Enrichment Score (ES) of the GSEA method is used to calculate the level of enrichment for a particular gene set or pathway, if a gene is involved in the composition of a pathway or gene set, the ES score increases, otherwise it decreases, the Normalized Enrichment Score (NES) is normalized to the size of the pathway or gene set in question, a positive NES indicates the level of enrichment at the top of the rankings, a negative NES indicates the level of enrichment at the bottom of the rankings; single-sample gene set enrichment analysis (ssGSEA) is performed using the R package “GSVA” (v1.42.0), and gene ontology (GO) bioprocess gene sets are enriched by ClueGO (a Cytoscape software plug-in), followed by application of unsupervised consistent clustering analysis to the molecular data of SCLC tumor samples so as to identify potential molecular subtypes with clusters ranging from 2 to 5; Specifically, 80% item resampling (pItem), 1000 “reps” resampling, the “cluster Alg” k-means method, and the Euclidean “distance” are chosen as key input parameters for the consensus clustering model, and the best consistent clustering k is selected using the cumulative distribution function (CDF) reference;
step 5, construction of CCI analysis model:
the CCI analysis model is constructed using two methods based on genetic characterization of immune cells, which include the xCell method and the ssGSEA method; specifically, the xCell method is used to assess the infiltration of 64 immune cells and stromal cell types, and then the Extreme Gradient Enhancement Machine Learning algorithm is used to construct the CCI analysis model, and the core function of the CCI analysis model is the binary logic, with a maximum augmentation iteration of 3,000; in order to ensure the robustness of the model, the depth of the tree is considered to be 4, and the proportion of training instances and columns sub-sampled in each tree is 50%, while the evaluation metrics of the validation data are displayed on the CCI analytical model using the “error” display, i.e., in this embodiment, the CCI analytical model modelled by machine learning shows the best trade-off between the predictive performance and the model complexity; in the case of a CCI (2-chemokine signature) specified on the 0-1 index, the upper threshold in the training cohort is defined as 0.4;
step 6, result verification:
the validation sample set in step 1, that is, the other 30 sets, is used as a baseline to measure protein expressions of CCL5 and CXCL9 using quantitative computerized IHC analysis for experimental validation at a protein level, and the CCI analysis models in step 5 are analytically validated in terms of stability and reliability; the CCI analysis model is further characterized for prognostic value in traditional SCLC subtypes by performing stratified analyses in a meta-cohort; and the prognostic value of the CCI analysis model in traditional SCLC subtypes is further characterized by stratifying the analysis in a meta-cohort (N=145); and step 7, typing determination:
based on results of step 6, the CCI analysis models are confirmed in terms of stability and reliability, then the CCI analysis model is applied to a new SCLC immunophenotyping.

In conjunction with Embodiments 1 and 2, the present application uses unsupervised hierarchical clustering to identify patterns of co-expression and biological activity of predefined genes; to further characterize the cellular and functional properties of the TME, the present application uses ssGSEA to score 29 FGES from the WTA mRNA expression profiles, and the unsupervised hierarchical clustering analysis of the 29 FGES classifies the 29 SCLC samples into two clusters with significantly different immune compartments, with one cluster characterized by a higher level of immune infiltration called the immune-enriched subtype (IE subtype), and the other called the and immune-deprived subtype (ID subtype).

The present application uses multidimensional analyses of RNA sequencing and protein quantification to identify IE subtype and ID subtype characterized by different immune profiles and clinically different prognoses and therapeutic outcomes, and, specifically, the present application constructs a CCI analytical model to differentiate between IE subtypes and ID subtypes by IHC, which is of great potential for risk stratification of patients and selection of beneficiaries for immunotherapy.

The immune classification of the IE and ID subtypes allows for further stratification of patient survival and patient response to chemotherapy or chemotherapy plus immunotherapy, and the immune classification of the present application outperforms the traditional NE and TF subtypes in differentiating prognosis and response to treatment.

Neither NE nor TF subtypes fully differentiate the immune status of small-cell lung cancer compared to the immune subtypes, although small-cell lung cancers with low NE are associated with increased immune cell infiltration (i.e. CD45+, CD3+, and CD8+ cells), which can be referred to as a “hot” or “immune oasis” phenotype in comparison with NE-high tumors with an “immune desert” phenotype. The immune subtype of the present application is unique in that it can distinguish the prognosis of each of the subgroups of NE and TF with better adaptability and robustness than the traditional subgroups of NE and TF.

The present application adopts the whole transcriptome and protein digital spatial conformation to analyze the microenvironmental characteristics of SCLC at the RNA and protein levels, respectively, and puts forward the concept of immunophenotyping to classify SCLC into immune-enriched (IE) and immune-deprived (ID) subtypes, and determines and verifies the CCL5/CXCL9 index (CCI) as a predictor of the above mentioned immunophenotypes with the support of machine learning algorithms, so that by setting the CCI exponent threshold of 0.4, SCLC is classified into IE and ID subtypes with better prognosis and more effective to immunotherapy, whereas ID subtypes have poor prognosis and are less effective to immunotherapy. The CCI analysis model constructed by the present application serves as a clinical guide for risk stratification of patients and selection of immunotherapy regimens.

The above illustrates and describes the basic principles, main features and advantages of the present application. It is to be understood by those skilled in the art that the present application is not limited by the above embodiments, and that the above embodiments and the description in the specification are merely illustrative of the principles of the present application. Without departing from the spirit and the scope of the present application, various modifications and improvements are still possible, and these modifications and improvements fall within the scope of the present application claimed to be protected. The scope of the claimed protection of the present application is defined by the appended claims and their equivalents.

1. An immunophenotyping method for small-cell lung cancer based on multidimensional analyses, comprising following steps:
step 1, sample collection:
several sets of formalin-fixed and paraffin-embedded tumor tissues of resected small-cell lung cancer are collected from archived electronic medical record system of a hospital as study samples, and the study samples collected are evenly divided into an analytical sample set as well as a validation sample set;
step 2, RNA extraction:
based on the analytical sample set, on each of its sample blocks, several sets of sections are cut and total RNA is isolated from the sections using a paraffin-embedded tissue total RNA extraction kit, and then quantified using a spectrophotometer along with a bioanalyzer for quality control, followed by extraction of RNA from each sample;
step 3, RNA sequencing:
tissue microarrays are constructed from small-cell lung cancer resected from the analytical sample set, by selecting two representative regions of tumor tissue per case, followed by sequencing on an illumination sequencing platform;
step 4, unsupervised hierarchical clustering analysis:
several sets of multicentered small-cell lung cancer patient cohorts are collected from high-throughput gene expression and corresponding publications to obtain clinicopathological information on small-cell lung cancer patients in the small-cell lung cancer patient cohorts, followed by functional and gene enrichment analyses, then unsupervised coherent clustering analysis is applied to molecular data from small-cell lung cancer tumor samples, and potential molecular subtypes with clustering numbers of 2-5 are identified;
step 5, construction of cell based computational (CCI) analysis model:
the CCL5 and CXCL9 index (CCI) analysis model is constructed using two methods based on genetic characterization of immune cells, including xCell method and ssGSEA method, and then CCI analysis model is constructed using extreme gradient augmented machine learning algorithms with an upper threshold in a pre-defined training cohort in a case of the CCI analysis model;
step 6, result verification:
the validation sample set in step 1 is used as a baseline to measure protein expressions of CCL5 and CXCL9 using quantitative computerized immunohistochemistry (IHC) analysis for experimental validation at a protein level, and the CCI analysis model in step 5 is analytically validated with respect to validation parameters; and
step 7, typing determination:
based on results of the step 6, the CCI analysis model is confirmed in terms of the validation parameters, and the CCI analysis model is applied to tumor tissues of a new small-cell lung cancer,
wherein the CCI analysis model is constructed by performing clustering of the tumor tissues of the untreated small-cell lung cancer, to identify patterns related with co-expression and biological activity of pre-defined genes associated with the tumor tissues.

2. The immunophenotyping method for small-cell lung cancer established based on multidimensional analyses according to claim 1, wherein in the step 1, an inclusion criterion for sample analysis is: after radical surgery coupled with systemic lymph node dissection, histologically confirmed as small-cell lung cancer without a component of composite non-small-cell lung cancer, no history of other malignant tumors, and no coexisting tumors in other organs.

3. The immunophenotyping method for small-cell lung cancer established based on multidimensional analyses according to claim 1, wherein in the step 2, additional tissue sections of each sample are subjected to hematoxylin and eosin staining for pathological verification of tumor areas and borders for macroscopic dissection prior to RNA extraction.

4. (canceled)

5. The immunophenotyping method for small-cell lung cancer established based on multidimensional analyses according to claim 1, wherein in the step 3, data obtained are subjected to quality control (QC) checking and normalization with a quality control normalization method.

6. The immunophenotyping method for small-cell lung cancer established based on multidimensional analyses according to claim 1, wherein in the step 5, the CCI analysis model has a core function of binary logic with a pre-defined augmentation iteration based on CCL5 and CXCL9 expression.

7. The immunophenotyping method for small-cell lung cancer established based on multidimensional analyses according to claim 1, wherein in the step 6, the CCI analysis model classifies small-cell lung cancer cases into high CCI group and low CCI group and uses 0.4 as a threshold to represent immune-enriched subtype (IE subtype) and immune-deprived subtype (ID subtype).

8. The immunophenotyping method for small-cell lung cancer established based on multidimensional analyses according to claim 1, wherein in the step 6, the CCI analysis model is further characterized for calculation of prognostic value in traditional small-cell lung cancer subtypes by performing stratified analyses in a meta-cohort.
